(19) United States
(12) Patent Application Publication
(10) Pub. No.: US 2006/0136055 A1
(43) Pub. Date: Jun. 22, 2006

(54) Pseudoaccommodative equipment implanted for presbyopia correction

(76) Inventor: Francois Michel, Thonon Les Baines (FR)

Correspondence Address: YOUNG & THOMPSON, 745 SOUTH 23RD STREET, 2ND FLOOR, ARLINGTON, VA, 22202, US

(21) Appl. No.: 10/520,113

(22) PCT Filed: Apr. 29, 2003
(86) PCT No.: PCT/FR03/01333

§ 371 Date: Jan. 3, 2005

(30) Foreign Application Priority Data
Jul. 2, 2002 (FR) 02/08248

(51) Int. Cl. A61F 2/16

(52) U.S. Cl. 623/6.37

(57) ABSTRACT

An equipment includes two intraocular implants whose the optic part (7g, d) is provided, proximate its free end, with an actuating elements (10g, d) for varying the length of the edge in response to a control signal (Sc); two pressure sensors (4d, 4g) located between the eye balls and the insertion point either of the external rectus muscles or of the internal rectus muscles, for measuring each a pressure and transforming it into a pressure signal; a comparator for comparing the pressure signals and, if they fulfil a predetermined condition, in sending a condition fulfillment signal (Scs) to a relay (5d, 5d) each associated with one implant; and two such relays (5d, 5g) for sending, on reception of the signal (Scs), a control signal (Sc) to the actuating elements (10) of its associated implant.

The present invention concerns the correction of presbyopia.

In young persons with normal vision, the crystalline lens acts like a convex lens with variable focal distance and adapts its power to the distance of the object observed so that the image forms on the retina, this phenomenon being known as accommodation. Thus, if the object is far off, the image forms on the retina when the crystalline lens is relatively flat, at rest; by contrast, if the object is near or very near, the crystalline lens has to shorten its focal distance by arching, so that the image of the object can continue to form on the retina.

With age, generally around 45 years of age, the crystalline lens enlarges and no longer has the space it needs to be able to change sufficiently in geometry to ensure near vision. It is therefore necessary to provide it with some corrective means, the most common of which is a pair of spectacles for near vision, with monofocal, bifocal, multifocal or progressive lenses.

In addition, the crystalline lens may become opaque (a condition known as “cataract”) to the point of seriously impairing vision and having to be removed in order to restore the passage of light rays. The patient, thus rendered aphakic, is in most cases fitted with an intraocular lens, called an “implant”, to ensure formation, on the retina, of the images of far-off objects. Such intraocular implants comprise an optic part, fairly similar to a contact lens, from which “arms”, called haptics, project and serve to fix the implant in the eye. An aphakic patient with this implant can no longer achieve any accommodation at all and requires spectacles for intermediate and near vision.

Various attempts have been made to remedy the partial or complete loss of accommodation without having to use progressive contact lenses or spectacles.

Thus, various surgical techniques have been proposed which are not reserved to aphakic patients:

for phakic or aphakic patients, placement of a diffractive or multifocal implant (in front of the iris or in the lens sac) aimed at replacing progressive spectacle lenses;

for aphakic patients, placement of an implant in the lens sac, “hinged” at the junction between the part of the implant and the haptic element and having to permit a slight anteroposterior movement to and fro;

for phakic patients, placement of scleral expansion bands in an attempt to restore to the crystalline lens the space necessary for its changes of shape during accommodation;

for phakic subjects, shaping of the cornea by excimer laser with the aim of bringing zones of different power into contiguity and thereby generating clear images and blurred images depending on the distance of the object observed, images which the brain is required to select or neutralize, respectively.

All these techniques have proven imperfect, unsatisfactory, or even ineffective, for the following reasons:

The multifocal implant assumes that the brain is able to permanently select between a clear image and a blurred image and neutralize the latter, which is in fact very uncertain. Moreover, the implant does not have power increments covering all distances to procure clear vision from 30 cm to far off.

The hinged implant is based on the conviction that the system of ciliary body, zonule and lens capsule remains effective and that it will exert a greater or lesser pressure on the haptics, bringing about a translation on the anteroposterior axis of the optic. In this case too, the clinical results are highly uncertain, difficult to reproduce, and subject to some doubt as to their duration, given the changes in the system of ciliary body, zonule and lens capsule.

Scleral expansion bands have proven ineffective.

As regards shaping of the cornea by laser, which is claimed to afford a refractive solution at a time instant “t”, this is by definition destined to have to be repeated in even the best of cases. It also assumes a cerebral plasticity.

The object of the present invention is to overcome the disadvantages of the aforementioned techniques and, to do this, it is based on a novel approach to the problem of restoring accommodation, both in phakic patients and in aphakic patients.

The invention is based on the fact that accommodation is indissociable from convergence. These two phenomena are linked by the same innervation and constitute the “accommodation-convergence” reflex.

In a patient with normal vision, the accommodative transformation of the crystalline lens is triggered by the perception, on the retina, of a blurred image generated by the observer observing a near object; the nearer the object, the more the crystalline lens accommodates. At the same time, to see this near object, the observer has to turn his gaze on it, and there will be increasing convergence the nearer the object.

The invention exploits this interrelationship between accommodation and convergence. More precisely, the invention uses convergence as a means of controlling the geometry of the optic part of an intraocular implant.

The state of convergence, however, cannot be used directly. To identify a state of convergence and a degree of convergence, the invention uses the pressure exerted either by the external rectus muscles on the eyeballs, when the internal rectus muscles contract, or the pressure exerted on the eyeballs by the contracted internal rectus muscles.

More precisely, each time an eye turns inward, it does so under the effect of the contraction of the internal rectus muscle. During the rotation movement, the insertion of the external rectus muscle is projected forward, pressing the end of the muscle body and the tendon against the eyeball. Pressure is thus exerted on the eyeball by the external rectus muscle. Likewise, pressure is exerted on the eyeball by the internal rectus muscle.

To achieve convergence, it is necessary for both eyes to turn inward and thus, in the case of convergence, pressure is exerted simultaneously on the respective eyeball by the two external rectus muscles, and by the two internal rectus muscles. It is the simultaneity of external pressure on the two eyeballs, or the simultaneity of internal pressure on said eyeballs, that reveals the state of convergence because pressure, either internal or external, on only one of the two eyes will not indicate convergence but the fact that the right eye is looking to the left, or the left eye to the right.

Thus, the invention is based on a method of temporary arching of a flexible piece approximately in the shape of a spherical cap, in this instance the optic part of an intraocular implant, which method involves:

providing said flexible piece, near its free edge, with an actuating means for varying the length of said free edge;

measuring the pressure at at least two points distant from one another, in this instance between each of the external rectus muscles (or each of the internal rectus muscles) and the associated eyeball, and converting each measured pressure into a pressure signal;

comparing said pressure signals coming from said two points, and

if they satisfy a predetermined relationship, in this instance simultaneity, sending a control signal acting on said actuating means for the purpose of modifying the length of the free edge of said piece and hence the radius or radii of curvature of the spherical cap.

In the application to restoring accommodation, the method according to the invention consists in sending said control signal if the comparison of said pressure signals reveals a simultaneity of increasing pressure at said two distant points (in this instance a state of increasing convergence), in which case the control signal acts on said actuating means to reduce the radius or radii of curvature of said spherical cap, that is to say the optic part of the intraocular implant which thus reduces its focal distance, or a simultaneity of decreasing pressure at said two distant points (in this instance a state of decreasing convergence), in which case the control signal acts on said actuating means to increase the radius or radii of curvature of said spherical cap, that is to say the optic part of the intraocular implant which thus increases its focal distance.

Naturally, during a period of stable simultaneous pressure, the control signal keeps the state of the actuating means stable.

The intraocular implant can thus behave in a manner similar to that of a natural and normal crystalline lens, such that the intraocular implant according to the invention can be considered as “pseudo-accommodative”.

Each pressure signal is preferably proportional to the measured pressure, in such a way as to adjust the pseudo-accommodation as a function of the degree of convergence and, in practice, the control signal is proportional to the mean of the two pressure signals satisfying the predetermined condition.

The invention consequently concerns optic equipment of the type comprising two intraocular implants, each composed of a flexible optic part approximately in the shape of a spherical cap, and of haptics for immobilizing said implant in place, characterized in that it comprises:

two such implants whose optic part is provided, near its free edge, with an actuating means for varying the length of said edge in response to a control signal;

two pressure sensors situated at a distance from one another, in this instance between the insertion of the external rectus muscle (or of the internal rectus muscle) and the eyeball, and each designed to measure a pressure and to convert it into a pressure signal;

a comparator designed to compare the pressure signals generated by the two sensors and, if they satisfy a predetermined condition, to send a “condition satisfied” signal to a relay associated with each implant; and

two such relays which are each designed to send, on receipt of a “condition satisfied” signal, a control signal to the actuating means of its associated implant.

The comparator can be a means distinct from the pressure sensors, but in a preferred embodiment each pressure sensor at one and the same time performs the function of a device for measuring the pressure at the point where it is situated, the function of comparing the pressure it measures with the pressure measured by the other pressure sensor, and, if the condition is satisfied, the function of transmitting the “condition satisfied” signal.

The sensors are preferably remote-powered electronic components and teletransmit the pressure measurement signals and, where appropriate, the “condition satisfied” signals.

Likewise, said relay or relays are remote-powered electronic components and teletransmit the control signals on receipt of a “condition satisfied” signal.

In one practical embodiment, each actuating means can comprise a filament of material of variable length attached to the periphery of the free edge of the optic part of an implant, and a device designed to modify the length of said filament, said device, which is remote-powered, being remote-controlled via one of said relays.

The invention moreover extends its scope to an intraocular implant composed of a flexible optic part approximately in the shape of a spherical cap, and of haptics for immobilizing it in place, characterized in that it comprises an actuating means comprising a filament of material of variable length attached to the periphery of the free edge of said optic part, and a device designed to modify the length of said filament, said device being designed to be remote-powered and to be remote-controlled.

The invention moreover extends its scope to a method for correcting presbyopia in a patient by means of the optic equipment as defined above, which method involves fitting one of said implants in each of the patient's eyes, either in the emptied lens sac of the aphakic patient, or in the anterior chamber of the phakic patient, and inserting a pressure sensor between each of the external rectus muscles (or each of the internal rectus muscles) and the associated eyeball.

The invention will be described in more detail below with reference to the attached drawings, in which:

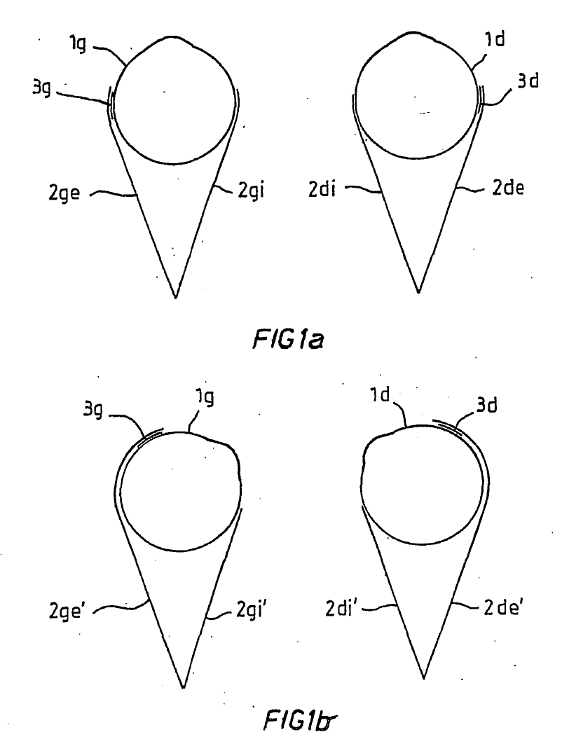

FIGS. 1a and 1b are schematic representations of the two eyeballs of a patient, respectively in near vision and in far vision, with their rectus muscles and the placement of the pressure sensors, in one possible embodiment of the invention;

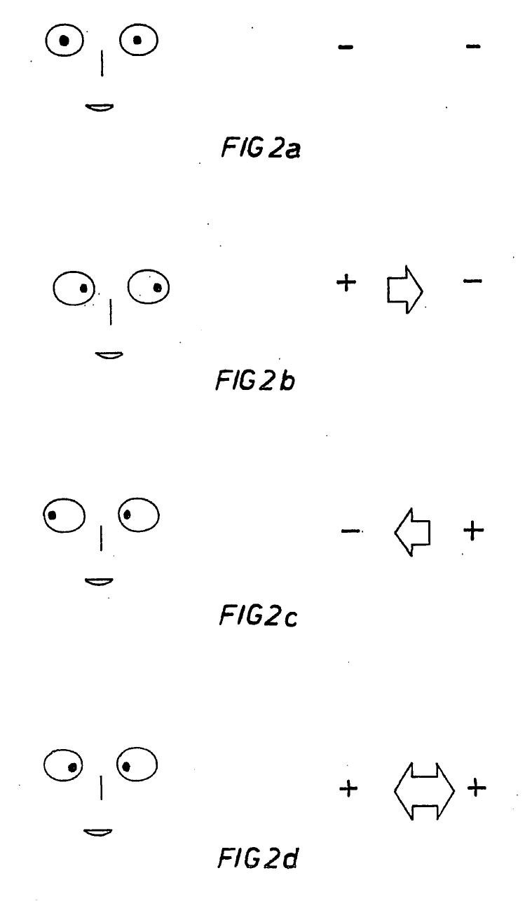

FIGS. 2a-d show various positions of a patient's eyes and, in parallel, the translation in terms of pressure detection;

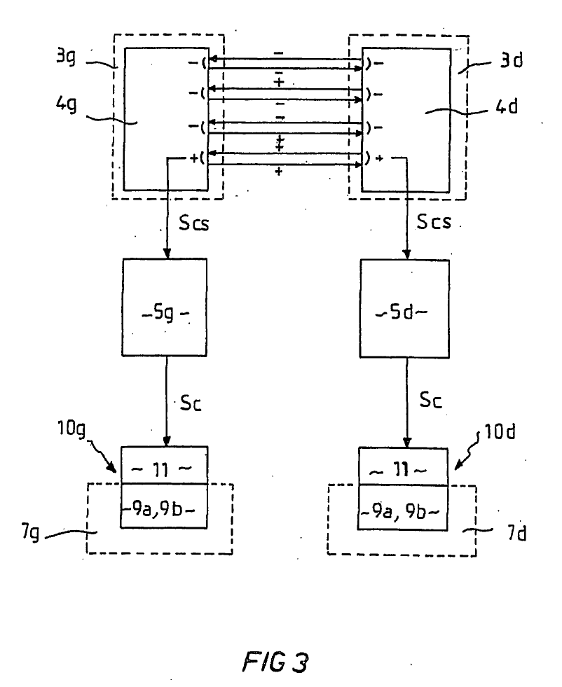

FIG. 3 is a block diagram explaining the method according to the invention;

FIG. 4 is a schematic representation of an intraocular implant according to the invention; and

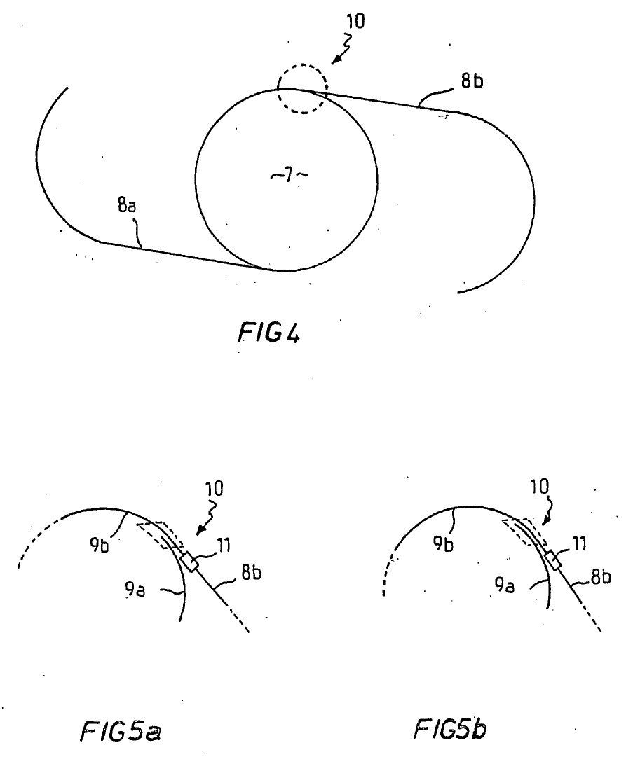

FIGS. 5a and 5b show, on a larger scale, the overlap zone of the strands of the filament surrounding the optic part of the implant according to the invention, respectively in far vision and in near vision.

Referring to FIG. 1a, this shows the two eyeballs 1d and 1g of a patient with their respective internal and external rectus muscles 2de, 2di and 2ge, 2gi, none of which is contracted, so that the eyes are looking straight in front, in far vision.

To observe a near object, the patient has to achieve convergence and, to do this, has to turn the right eye toward the left and the left eye toward the right. To this end, the internal rectus muscles 2di′, 2gi′ contract, forcing the eyeball to turn, and this means that the insertions of the external rectus muscles 2de′, 2ge′ project forward, pressing the end of the muscular body and the tendon against their respective eyeball, as is shown in FIG. 1b. In doing this, the muscles 2de′, 2ge′ exert a pressure on their eyeball, which pressure can be detected and quantified by placing a suitable device in the zone 3d, 3g under the tendon of insertion of the muscle.

FIGS. 2a-2d show, in parallel, the position of the patient's eyes and the detection or non-detection of pressure.

In FIG. 2a, the patient is looking to the front, as in FIG. 1a. No pressure is exerted at 3d or 3g.

In FIG. 2b, the patient is looking to the left: pressure is exerted at area 3d (FIG. 1b), but not at area 3g (FIG. 1b).

In FIG. 2c, the patient is looking to the right: pressure is exerted at area 3g (FIG. 1b), but not at area 3d (FIG. 1b).

In FIG. 2c, the patient achieves convergence: pressure is exerted simultaneously at area 3g and at area 3d. It is this simultaneity which indicates the state of convergence. In the absence of simultaneity, the method and equipment according to the invention remain inactive.

FIG. 3 illustrates the principle of the method according to the invention. Reference numbers 4d and 4g indicate strain gages which can be formed from miniature absolute pressure sensors inserted, as indicated above, at 3d and 3g (see FIGS. 1a and 1d) under the tendon of insertion of the external rectus muscles. These can be microstructures on silicon, of the order of a millimeter across, which are powered without contacts and without batteries, for example by induction. Such systems comprise a sensitive element, a converter, and a coupler connected to a secondary antenna permitting remote powering of the system and teletransmission of the pressure measurement.

More precisely, the sensitive element is a mechanical microstructure that deforms under the effect of a force, in this instance the pressure to which it is subjected, which deformation causes modification of capacitances integrated in the sensitive unit. The electric value of the capacitance variations is converted into a digital signal by the converter, and this digitized pressure signal is transmitted to the other strain gage, and vice versa, for comparison purposes. To do this, an external magnetic field powers the converter, via the secondary antenna, and the digitized pressure signals are transmitted from one strain gage to the other through modulation of said magnetic field.

The strain gages 4d and 4g are thus capable of detecting and of quantifying the pressure to which they are subjected and of communicating their pressure information to one another.

With reference to FIGS. 3a-3d, this communication may be non-existent (−/−), in which case nothing happens. It may also be unilateral (± or ∓), in which case, once again, nothing happens. It is only when it is mutual and simultaneous (+/+) that each gage establishes that there is a state of convergence and sends a “condition satisfied” signal Scs to a respective electronic relay 5d and 5g. Each “condition satisfied” signal Scs is proportional to the pressure measured at each given instant by the strain gage 4d, 4g concerned, or, better still, proportional to the mean of the pressures measured by the two strain gages 4d, 4g at each given instant. It follows that the signal Scs can reflect a state of greater or lesser convergence, depending on the distance of the object in near vision, or of increasing convergence if the object is coming closer, and also a state of decreasing convergence (return to far vision).

Each relay 5d, 5g sends a control signal Sc, proportional to the “condition satisfied” signal Scs, to an actuator 10d, 10g comprising an open-loop filament 9a, 9b which surrounds respectively the optic part 7d, 7g of a right intraocular implant and of a left intraocular implant, and which is designed to modify the radii of curvature of said optic part and, consequently, the power of said optic part, under the effect of a device 11 included in the actuator 10.

The device 11 in question also preferably takes the form of a microsystem operating without contacts and devoid of any battery. This microsystem comprises a mechanical part, as will be seen below, and a coupler of the radio-frequency type connected to a secondary antenna permitting remote powering of the microsystem and reception of the control signals.

In one embodiment of the invention, the electronic relays 5d and 5g are built into a spectacle frame, as are, likewise, microbatteries which power the strain gages 4d, 4g and the actuators 10d, 10g. The spectacle frame also comprises four primary antennas which generate the magnetic field needed for powering the two strain gages 4d, 4g and the two actuators 1d, 10g, and a computer for digitizing the pressure measurements conducted by the strain gages, for the purpose of generating a signal Sc proportional to said pressure measurements for transmission to the actuators.

Referring now to FIG. 4, this shows a schematic representation of a pseudo-accommodative intraocular implant according to the invention. In a manner known per se, the implant comprises an optic part 7 and haptics 8a, 8b. According to the invention, the optic part 7 is surrounded by a filament of material in the form of an open loop which is closed on itself by overlapping of its strands, and which is laid in a groove provided on the periphery of the optic part. The length of this surround is variable depending on the degree of overlap of the stands of the filament loop, as is shown in FIGS. 5a and 5b. Thus, in the absence of accommodation, the overlap between the strands 9a, 9b is minimal whereas, in the case of accommodation, this overlap is more pronounced the greater the required accommodation (in other words the control signal Sc reflects a higher pressure in the area of the strain gages 4d, 4g). The more pronounced the overlap, the more the perimeter of the optic part 7 is reduced and the more the two faces of this optic part bulge, with a corresponding increase in power.

For an implant of 6 mm in diameter, or a perimeter of 18.84 mm, a decrease of 1.82 mm of the perimeter corresponds to an increase of 3 diopters of refractive power (representing submaximal accommodation, with maximum accommodation being 3.5 diopters).

To do this, one of the strands of the loop is continued by one of the haptics 8b, the latter comprising at its proximal end, that is to say immediately adjacent to the optic part 7, an electrostatic actuator 10 whose maximum movement corresponds to the maximum range of variation of the perimeter of the surround of the optic part 7. A secondary antenna is provided on the haptic 8b in order to receive the modulations of the magnetic field carrying the control signal Sc that acts on the actuator 10.

The pseudo-accommodative implant according to the invention will be placed either in the lens sac emptied of its contents in cataract surgery (aphakic patient) or in the anterior chamber (that is to say in front of the iris) in the phakic patient.

In terms of surgery, all the maneuvers used for implementing the method are traditional ones. The cataract operation is standardized, likewise the placement of an implant in the capsular bag. The insertion of an implant in the anterior chamber of a phakic patient is also a well-established procedure. As regards the fitting of a strain gage under the external rectus muscle, this calls on the surgical technique used to treat strabismus.

It will be appreciated that the invention is not limited to the embodiment that has been described and shown. In particular, instead of being implanted between the point of insertion of the external rectus muscles and the eyeball as shown in FIGS. 1a and 1b, the pressure sensors could be implanted between the insertion of the internal rectus muscles and the eyeball. Moreover, instead of powering all the components by means of a battery placed in a spectacle frame, it will no doubt be possible, in the near future, and by virtue of the miniaturization of the elements and the use of rechargeable implanted microbatteries, to fit all the necessary equipment in and around the patient's eye.

1-7. (canceled)

8. An optic equipment of the type comprising two intraocular implants, each composed of a flexible optic part approximately in the shape of a spherical cap, and of haptics for immobilizing said implant in place, wherein:
two such implants whose optic part is provided, near its free edge, with an actuating means for varying the length of said edge in response to a control signal;
two pressure sensors situated at a distance from one another and each designed to measure a pressure and to convert it into a pressure signal;
a comparator designed to compare the pressure signals generated by the two sensors and, if they satisfy a predetermined condition, to send a “condition satisfied” signal to a relay associated with each implant; and
two such relays which are each designed to send, on receipt of a “condition satisfied” signal, a control signal to the actuating means of its associated implant.

9. The equipment as claimed in claim 8, wherein each pressure sensor at one and the same time performs the function of a device for measuring the pressure at the point where it is situated, the function of comparing the pressure it measures with the pressure measured by the other pressure sensor, and, if the condition is satisfied, the function of transmitting the “condition satisfied” signal.

10. The equipment as claimed in claim 8, wherein said sensors are remote-powered electronic components and teletransmit the pressure measurement signals and, where appropriate, the “condition satisfied” signals.

11. The equipment as claimed in claim 8, wherein said relay or relays are remote-powered electronic components and teletransmit the control signals on receipt of a “condition satisfied” signal.

12. The equipment as claimed in claim 8, wherein each actuating means comprises a filament of material of variable length attached to the periphery of the free edge of the optic part of an implant, and a device designed to modify the length of said filament, said device, which is remote-powered, being remote-controlled via one of said relays.

13. An intraocular implant composed of a flexible optic part approximately in the shape of a spherical cap, and of haptics for immobilizing it in place, wherein it comprises an actuating means comprising a filament of material of variable length attached to the periphery of the free edge of said optic part, and a device designed to modify the length of said filament, said device being designed to be remote-powered and to be remote-controlled.

14. Method for correcting presbyopia in a patient by means of the optic equipment as claimed in claim 8, wherein it involves fitting one of said implants in each of the patient's eyes, either in the emptied lens sac of the aphakic patient or in the anterior chamber of the phakic patient, and inserting a pressure sensor between each of the external rectus muscles (or each of the internal rectus muscles) and the associated eyeball.
